(19) United States
(12) Patent Application Publication
(10) Pub. No.: US 2020/0361863 A1
(43) Pub. Date: Nov. 19, 2020

(54) METHOD OF SYNTHESIZING ALPHA-AMINO ACID DERIVATIVES

(71) Applicant: Nanjing Tech University, Nanjing (CN)

(72) Inventors: Yaojia JIANG, Nanjing (CN); Yang CHEN, Nanjing (CN); Teck-Peng Loh, Penang (MY)

(21) Appl. No.: 16/855,359

(22) Filed: Apr. 22, 2020

(30) Foreign Application Priority Data
May 13, 2019 (CN) 201910394505.3

(51) Int. Cl. C07C 319/06

(52) U.S. Cl. CPC C07C 319/06

(57) ABSTRACT

Disclosed is a method of synthesizing an α-amino acid derivative, where an azirine compound, used as raw material, is reacted with a sulfhydryl compound in a mixed system of an organic solvent and a buffer solution under the protection of nitrogen to produce the α-amino acid derivative. In the reaction, the carbon-nitrogen double bond of the azirine compound is attacked by the sulfhydryl compound, so that the azirine compound is directly opened to form the α-amino acid derivative of which the same carbon atom is added with two molecules of the sulfhydryl compound. This method can be performed directly in an aqueous phase without using a metal catalyst.

### CROSS-REFERENCE TO RELATED APPLICATIONS

This application claims the benefit of priority from Chinese Patent Application No. 201910394505.3, filed on May 13, 2019. The content of the aforementioned application, including any intervening amendments thereto, is incorporated herein by reference in its entirety.

### TECHNICAL FIELD

This application relates to organic synthesis, and more particularly to a method of synthesizing α-amino acid derivatives.

### BACKGROUND

α-amino acids are the primary components of proteins and the most important amino acids in organisms. In addition, they are also one of the most important and generic components for biological and chemical synthesis. Currently, there are several methods available for totally synthesizing amino acids, for example, an enamide and an α-enantioselectively hydrogenated glycine synthon are subjected to alkylation to produce α-amino acids; and based on the reaction between an aromatic aldehyde and :CCl₂ (dichlorocarbene), an aromatic aldehyde is allowed to react with chloroform, potassium hydroxide, lithium chloride and ammonia in the presence of a phase transfer catalyst to produce an α-amino acid. However, these methods often have complex process and long reaction period, moreover, these methods also require a complete organic phase system and a metal catalyst.

Azirine has a three-membered ring structure containing one nitrogen atom and two carbon atoms, where the nitrogen atom bonds to one of the two carbon atoms through a double bond. Azirine is the most stable unsaturated three-membered nitrogen-containing heterocycle. The chemical activity of azirine is closely associated with the high-strength three-membered ring, which improves the reactivity of the carbon-nitrogen double bond and facilitates the ring-opening reaction. Azirine compounds are a kind of reactive reagents and can be used as nucleophilic reagents, electrophilic reagents, dienophiles and dipolarophiles to participate in various chemical reactions, having broad application prospects in the organic synthesis, especially the construction of nitrogen-containing heterocyclic compounds. With the gradual improvement of the synthetic methods, the azirine compounds will be increasingly applied in the organic synthesis.

Neber et al. first report the azirine compounds in 1932. Azirine compounds are naturally-occurring antibiotics and have been found in many natural products, such as vitamins, hormones, antibiotics and alkaloids. Moreover, azirine compounds are also introduced in medicines, pesticides other important chemicals such as corrosion inhibitors, anti-aging drugs, sensitizers and stabilizers. Azithromycin is a class of azirine compounds isolated from Streptomyces aureofaciens.

The ring-opening reaction for the azirine compounds has been extensively investigated, including the breaking of carbon-nitrogen single bond and carbon-carbon single bond, but it has been hardly reported about the breaking of carbon-nitrogen double bond.

### SUMMARY

An object of this application is to provide a method of synthesizing an α-amino acid derivative to overcome the defects in the prior art.

The technical solutions of this application are described as follows. An azirine compound, used as raw material, is reacted with a sulfhydryl compound in a mixed system of an organic solvent and a buffer solution under the protection of nitrogen to produce the α-amino acid derivative, where the carbon-nitrogen double bond of the azirine compound is attacked by the sulfhydryl compound to allow the azirine compound to be directly opened to form the α-amino acid derivative, of which the same carbon atom is added with two molecules of the sulfhydryl compound. The method is specifically described below.

This application provides a method of synthesizing an α-amino acid derivative, comprising:

adding a solvent and a potassium dihydrogen phosphate-dipotassium hydrogen phosphate buffer solution having a pH of 6.24-7.40 to a reactor; adding an azirine compound; vacuumizing the reactor and introducing nitrogen; dropwise adding a sulfhydryl compound for reaction; desolventizing the reaction mixture; redissolving the reaction mixture with water; extracting the reaction mixture three times with ethyl acetate; combining the resulting organic phases; and subjecting the combined organic phase to drying, rotary evaporation and column chromatography to produce the α-amino acid derivative.

In an embodiment, a volume ratio of the potassium dihydrogen phosphate-dipotassium hydrogen phosphate buffer solution to the solvent is 1:1.

In an embodiment, the solvent is selected from methanol, tetrahydrofuran and acetonitrile.

In an embodiment, a molar ratio of the azirine compound to the sulfhydryl compound is 1:8.

In an embodiment, the azirine compound has the following formula:

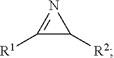

wherein:

R¹ is selected from Ph, 4-OMe-Ph and 4-Cl-Ph; and

R2 is selected from COOtBu, COONH₂ and H.

In an embodiment, the sulfhydryl compound is selected from thiophenol, 4-methoxythiophenol, 4-chlorothiophenol and 4-methylthiophenol.

In an embodiment, a temperature of the reaction is 20-40° C. and a time of the reaction is 15-30 h.

This application has the following beneficial effects.

The method provided herein for synthesizing an α-amino acid derivative can be directly performed in an aqueous phase without using a metal catalyst. Moreover, this method also has simple and mild reaction conditions and high yield.

### DETAILED DESCRIPTION OF EMBODIMENTS

The invention will be described in detail below with reference to the embodiments.

### Example 1

The entire reaction was shown as follows:

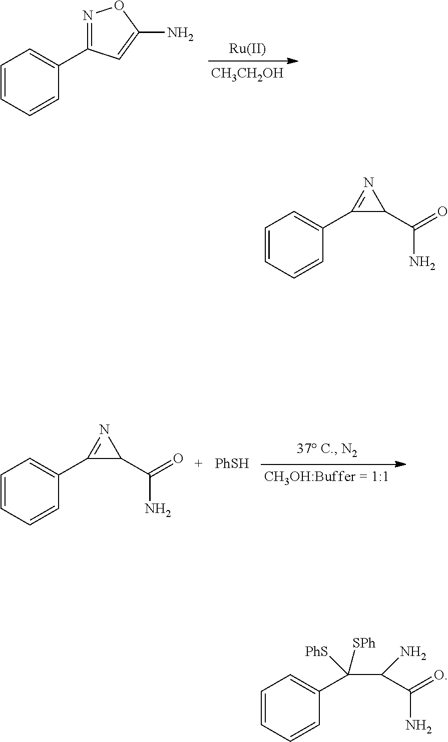

An amide-carrying azirine compound was prepared as follows. 1.0 equiv. of oxazole (0.2 mmol), 2 mL of analytical ethanol and 1% equiv. of a ruthenium catalyst were added to a Schlenk tube. The reaction mixture was reacted under green light for 24-72 h. After the reaction was completed, the reaction mixture was desolventized by rotary evaporation, redissolved with water and extracted three times with dichloromethane. The three organic phases were combined, dried with anhydrous sodium sulfate and then subjected to rotary evaporation and column chromatography to produce the amide-carrying azirine compound, where an eluent used in the column chromatography was a mixture of dichloromethane and methanol.

An α-amino acid derivative (bis(phenylthio) propionamide) was synthesized as follows. 1.0 equiv. of the above amide-carrying azirine compound (0.2 mmol), 1.5 mL of analytical methanol and 1.5 mL of a pH 7.4 potassium dihydrogen phosphate-dipotassium hydrogen phosphate buffer solution were added to a Schlenk tube. The Schlenk tube was vacuumized, to which 8.0 equiv. of thiophenol was dropwise added under nitrogen. The reaction mixture was reacted for 20 h. After the reaction was completed, the reaction mixture was dried using a rotary evaporator, redissolved with water and extracted three times with ethyl acetate. The organic phases were combined, dried with anhydrous sodium sulfate and then subjected to rotary evaporation and column chromatography to produce bis(phenylthio) propionamide (yield 90%), where an eluent used in the column chromatography was a mixture of dichloromethane and methanol.

### Example 2

The entire reaction was shown as follows:

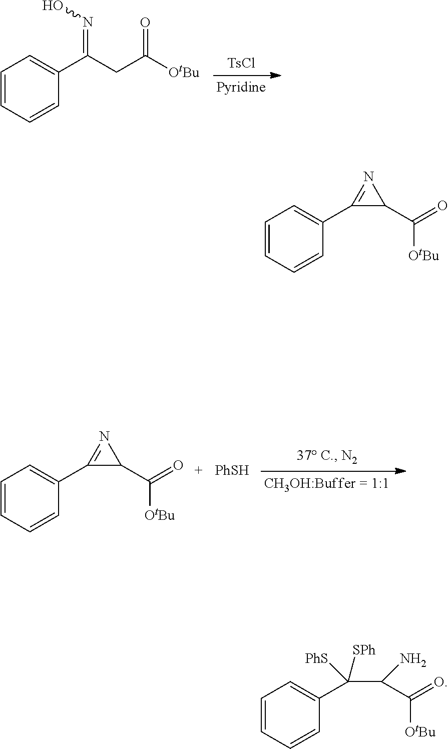

An ester group-carrying azirine compound was prepared as follows. 1.0 equiv. of N-tosylamine (0.2 mmol), 2 mL of analytical dichloromethane and 1.5 equiv. of triethylamine (0.3 mmol) were added to a Schlenk tube. The reaction mixture was reacted in an ice water bath under stirring for 6 h. The reaction was terminated as soon as the temperature was returned to room temperature. The reaction mixture was extracted three times with dichloromethane. The three organic phases were combined, dried with anhydrous sodium sulfate, rotarily evaporated to remove the excessive solvent and then subjected to column chromatography to produce the ester group-carrying azirine compound, where an eluent used in the column chromatography was a mixture of petrole ether and ethyl acetate.

An α-amino acid derivative (bis(phenylthio) propylamine t-butyl ester) was synthesized as follows. 1.0 equiv. of the above ester group-carrying azirine compound (0.2 mmol), 1.5 mL of analytical methanol and 1.5 mL of a pH 7.4 potassium dihydrogen phosphate-dipotassium hydrogen phosphate buffer solution were added to a Schlenk tube. The Schlenk tube was vacuumized, to which 8.0 equiv. of thiophenol was dropwise added under nitrogen. The reaction mixture was reacted for 20 h. After the reaction was completed, the reaction mixture was dried using a rotary evaporator and extracted three times with ethyl acetate. The organic phases were combined, dried with anhydrous sodium sulfate and then subjected to rotary evaporation and column chromatography to produce the bis(phenylthio) propylamine t-butyl ester (yield 86%), where an eluent used in the column chromatography was a mixture of dichloromethane and methanol.

### Example 3

The entire reaction was shown as follows:

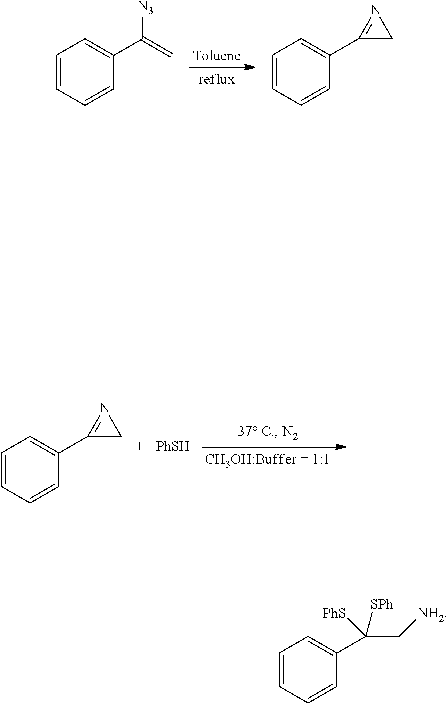

3-phenyl azirine compound was prepared as follows. 1.0 equiv. of 1-azido vinylbenzene derivative (0.2 mmol) was added to a round-bottomed flask to which 10 mL of toluene was added. The reaction mixture was refluxed at 110° C. for 6 h. After the reaction was completed, the reaction mixture was rotarily evaporated to remove the excessive solvent and then subjected to column chromatography to produce the 3-phenyl azirine compound, where an eluent used in the column chromatography was a mixture of petrole ether and ethyl acetate.

An α-amino acid derivative (bis(phenylthio) propylamine) was synthesized as follows. 1.0 equiv. of the above 3-phenyl azirine compound (0.2 mmol), 1.5 mL of analytical methanol and 1.5 mL of a pH 7.4 potassium dihydrogen phosphate-dipotassium hydrogen phosphate buffer solution were added to a Schlenk tube. The Schlenk tube was vacuumized, to which 8.0 equiv. of thiophenol was dropwise added under nitrogen. The reaction mixture was reacted for 20 h. After the reaction was completed, the reaction mixture was dried using a rotary evaporator and extracted three times with ethyl acetate. The organic phases were combined, dried with anhydrous sodium sulfate and then subjected to rotary evaporation and column chromatography to produce the bis(phenylthio) propylamine (yield 48%), where an eluent used in the column chromatography was a mixture of dichloromethane and methanol.

### Example 4

1.0 equiv. of the amide-carrying azirine compound (0.2 mmol) prepared in Example 1, 1.5 mL of analytical acetonitrile and 1.5 mL of a pH 6.98 potassium dihydrogen phosphate-dipotassium hydrogen phosphate buffer solution were added to a Schlenk tube. The Schlenk tube was vacuumized, to which 6.0 equiv. of thiophenol was dropwise added under nitrogen. The reaction mixture was reacted at 37° C. for 20 h. After the reaction was completed, the reaction mixture was dried using a rotary evaporator and extracted three times with ethyl acetate. The organic phases were combined, dried with anhydrous sodium sulfate and then subjected to rotary evaporation and column chromatography to give a product (yield 40%), where an eluent used in the column chromatography was a mixture of dichloromethane and methanol.

### Example 5

1.0 equiv. of the amide-carrying azirine compound (0.2 mmol) prepared in Example 1, 1.5 mL of analytical tetrahydrofuran and 1.5 mL of a pH 6.98 potassium dihydrogen phosphate-dipotassium hydrogen phosphate buffer solution were added to a Schlenk tube. The Schlenk tube was vacuumized, to which 6.0 equiv. of thiophenol was dropwise added under nitrogen. The reaction mixture was reacted at 37° C. for 20 h. After the reaction was completed, the reaction mixture was dried using a rotary evaporator and extracted three times with ethyl acetate. The organic phases were combined, dried with anhydrous sodium sulfate and then subjected to rotary evaporation and column chromatography to give a product (yield 60%), where an eluent used in the column chromatography was a mixture of dichloromethane and methanol.

### Example 6

1.0 equiv. of the amide-carrying azirine compound (0.2 mmol) prepared in Example 1, 1.5 mL of analytical methanol and 1.5 mL of a pH 6.98 potassium dihydrogen phosphate-dipotassium hydrogen phosphate buffer solution were added to a Schlenk tube. The Schlenk tube was vacuumized, to which 6.0 equiv. of thiophenol was dropwise added under nitrogen. The reaction mixture was reacted at 37° C. for 20 h. After the reaction was completed, the reaction mixture was dried using a rotary evaporator and extracted three times with ethyl acetate. The organic phases were combined, dried with anhydrous sodium sulfate and then subjected to rotary evaporation and column chromatography to give a product (yield 62%), where an eluent used in the column chromatography was a mixture of dichloromethane and methanol.

### Example 7

1.0 equiv. of the amide-carrying azirine compound (0.2 mmol) prepared in Example 1, 1.5 mL of analytical methanol and 1.5 mL of a pH 6.24 potassium dihydrogen phosphate-dipotassium hydrogen phosphate buffer solution were added to a Schlenk tube. The Schlenk tube was vacuumized, to which 6.0 equiv. of thiophenol was dropwise added under nitrogen. The reaction mixture was reacted at 37° C. for 20 h. After the reaction was completed, the reaction mixture was dried using a rotary evaporator and extracted three times with ethyl acetate. The organic phases were combined, dried with anhydrous sodium sulfate and then subjected to rotary evaporation and column chromatography to give a product (yield 64%), where an eluent used in the column chromatography was a mixture of dichloromethane and methanol.

### Example 8

1.0 equiv. of the amide-carrying azirine compound (0.2 mmol) prepared in Example 1, 1.5 mL of analytical methanol and 1.5 mL of a pH 7.40 potassium dihydrogen phosphate-dipotassium hydrogen phosphate buffer solution were added to a Schlenk tube. The Schlenk tube was vacuumized, to which 6.0 equiv. of thiophenol was dropwise added under nitrogen. The reaction mixture was reacted at 37° C. for 20 h. After the reaction was completed, the reaction mixture was dried using a rotary evaporator and extracted three times with ethyl acetate. The organic phases were combined, dried with anhydrous sodium sulfate and then subjected to rotary evaporation and column chromatography to give a product (yield 79%), where an eluent used in the column chromatography was a mixture of dichloromethane and methanol.

### Example 9

1.0 equiv. of the amide-carrying azirine compound (0.2 mmol) prepared in Example 1, 1.5 mL of analytical methanol and 1.5 mL of a pH 7.40 potassium dihydrogen phosphate-dipotassium hydrogen phosphate buffer solution were added to a Schlenk tube. The Schlenk tube was vacuumized, to which 4.0 equiv. of thiophenol was dropwise added under nitrogen. The reaction mixture was reacted at 37° C. for 20 h. After the reaction was completed, the reaction mixture was dried using a rotary evaporator and extracted three times with ethyl acetate. The organic phases were combined, dried with anhydrous sodium sulfate and then subjected to rotary evaporation and column chromatography to give a product (yield 86%), where an eluent used in the column chromatography was a mixture of dichloromethane and methanol.

### Example 10

1.0 equiv. of the amide-carrying azirine compound (0.2 mmol) prepared in Example 1, 1.5 mL of analytical methanol and 1.5 mL of a pH 7.40 potassium dihydrogen phosphate-dipotassium hydrogen phosphate buffer solution were added to a Schlenk tube. The Schlenk tube was vacuumized, to which 8.0 equiv. of thiophenol was dropwise added under nitrogen. The reaction mixture was reacted at 37° C. for 20 h. After the reaction was completed, the reaction mixture was dried using a rotary evaporator and extracted three times with ethyl acetate. The organic phases were combined, dried with anhydrous sodium sulfate and then subjected to rotary evaporation and column chromatography to give a product (yield 90%), where an eluent used in the column chromatography was a mixture of dichloromethane and methanol.

### Example 11

1.0 equiv. of the amide-carrying azirine compound (0.2 mmol) prepared in Example 1, 1.5 mL of analytical methanol and 1.5 mL of a pH 7.40 potassium dihydrogen phosphate-dipotassium hydrogen phosphate buffer solution were added to a Schlenk tube. The Schlenk tube was vacuumized, to which 10.0 equiv. of thiophenol was dropwise added under nitrogen. The reaction mixture was reacted at 37° C. for 20 h. After the reaction was completed, the reaction mixture was dried using a rotary evaporator and extracted three times with ethyl acetate. The organic phases were combined, dried with anhydrous sodium sulfate and then subjected to rotary evaporation and column chromatography to give a product (yield 68%), where an eluent used in the column chromatography was a mixture of dichloromethane and methanol.

### Example 12

1.0 equiv. of the amide-carrying azirine compound (0.2 mmol) prepared in Example 1, 1.5 mL of analytical methanol and 1.5 mL of a pH 7.40 potassium dihydrogen phosphate-dipotassium hydrogen phosphate buffer solution were added to a Schlenk tube. The Schlenk tube was vacuumized, to which 6.0 equiv. of thiophenol was dropwise added under nitrogen. The reaction mixture was reacted at 30° C. for 20 h. After the reaction was completed, the reaction mixture was dried using a rotary evaporator and extracted three times with ethyl acetate. The organic phases were combined, dried with anhydrous sodium sulfate and then subjected to rotary evaporation and column chromatography to give a product (yield 85%), where an eluent used in the column chromatography was a mixture of dichloromethane and methanol.

### Example 13

1.0 equiv. of the amide-carrying azirine compound (0.2 mmol) prepared in Example 1, 1.5 mL of analytical methanol and 1.5 mL of a pH 7.40 potassium dihydrogen phosphate-dipotassium hydrogen phosphate buffer solution were added to a Schlenk tube. The Schlenk tube was vacuumized, to which 6.0 equiv. of thiophenol was dropwise added under nitrogen. The reaction mixture was reacted at 20° C. for 20 h. After the reaction was completed, the reaction mixture was dried using a rotary evaporator and extracted three times with ethyl acetate. The organic phases were combined, dried with anhydrous sodium sulfate and then subjected to rotary evaporation and column chromatography to give a product (yield 88%), where an eluent used in the column chromatography was a mixture of dichloromethane and methanol.

What is claimed is:

1. A method of synthesizing an α-amino acid derivative, comprising:
adding a solvent and a potassium dihydrogen phosphate-dipotassium hydrogen phosphate buffer solution having a pH of 6.24-7.40 to a reactor;
adding an azirine compound to the reactor; vacuumizing the reactor and introducing nitrogen; dropwise adding a sulfhydryl compound for reaction;
desolventizing the reaction mixture; redissolving the reaction mixture with water;
extracting the reaction mixture three times with ethyl acetate; combining the resulting organic phases; and subjecting the combined organic phase to drying, rotary evaporation and column chromatography to produce the α-amino acid derivative.

2. The method of claim 1, wherein a volume ratio of the potassium dihydrogen phosphate-dipotassium hydrogen phosphate buffer solution to the solvent is 1:1.

3. The method of claim 1, wherein the solvent is selected from methanol, tetrahydrofuran and acetonitrile.

4. The method of claim 1, wherein a molar ratio of the azirine compound to the sulfhydryl compound is 1:8.

5. The method of claim 1, wherein the azirine compound has the following formula: 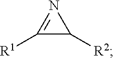
wherein:
R′ is selected from Ph, 4-OMe-Ph and 4-Cl-Ph; and
R² is selected from COOtBu, COONH₂ and H.

6. The method of claim 1, wherein the sulfhydryl compound is selected from thiophenol, 4-methoxythiophenol, 4-chlorothiophenol and 4-methylthiophenol.

7. The method of claim 2, wherein a temperature of the reaction is 20-40° C. and a time of the reaction is 15-30 h.

8. The method of claim 3, wherein a temperature of the reaction is 20-40° C. and a time of the reaction is 15-30 h.

9. The method of claim 4, wherein a temperature of the reaction is 20-40° C. and a time of the reaction is 15-30 h.

10. The method of claim 5, wherein a temperature of the reaction is 20-40° C. and a time of the reaction is 15-30 h.

11. The method of claim 6, wherein a temperature of the reaction is 20-40° C. and a time of the reaction is 15-30 h.
